(19) United States
(12) Patent Application Publication
(10) Pub. No.: US 2026/0040469 A1
(43) Pub. Date: Feb. 5, 2026

(54) CONDUCTIVE INJECTION MOLDED INTERCONNECT WITH PRINTED FLEXIBLE SUBSTRATE

(71) Applicant: Analog Devices, Inc., Wilmington, MA (US)

(72) Inventor: David Frank Bolognia, Charlestown, MA (US)

(21) Appl. No.: 19/288,851

(22) Filed: Aug. 1, 2025

Related U.S. Application Data
(60) Provisional application No. 63/678,179, filed on Aug. 1, 2024.

(51) Int. Cl. H05K 5/02; A61B 5/00; A61B 5/257; A61B 5/28; H05K 5/00

(52) U.S. Cl. CPC H05K 5/0247; A61B 5/257; A61B 5/28; A61B 5/6833; H05K 5/0086; A61B 2562/125

(57) ABSTRACT

An electronic device is disclosed. The electronic device can include a wearable interface having a first electrode and a second electrode spaced apart from the first electrode. The first electrode and the second electrode can be electrically connected by conductive traces. The electronic device can also include a first housing coupled to the wearable interface. The first housing can be coupled to an electronic component of the electronic device. The first housing can include a conductive bump configured to electrically contact the conductive traces. The first housing can also include a conductive post in electrical communication with the conductive bump. The conductive post can provide an electrical connection between a terminal of the electronic component and the first electrode and the second electrode.

### RELATED APPLICATIONS

This application claims the priority benefit of U.S. Provisional Patent Application 63/678,179 filed on Aug. 1, 2024, entitled “CONDUCTIVE INJECTION MOLDED INTERCONNECT WITH PRINTED FLEXIBLE SUBSTRATE,” which is incorporated by reference herein in its entirety.

### BACKGROUND

### Field

The field relates to a conductive injection molded interconnect with printed flexible substrate.

### Description of the Related Art

Wearable electronic devices are increasingly used by consumers to monitor various health characteristics, e.g., heart rate, etc. It can be challenging to package electronic components in wearable applications due to the environment of use, in which moisture and other contaminants are present. Accordingly, there is a continuing need for improved wearable devices.

### SUMMARY

For purposes of summarizing the disclosure and the advantages achieved over the prior art, certain objects and advantages of the disclosure are described herein. Not all such objects or advantages may be achieved in any particular implementation. Thus, for example, those skilled in the art will recognize that the devices, systems, and methods may be embodied or carried out in a manner that achieves or optimizes one advantage or group of advantages as taught herein without necessarily achieving other objects or advantages as may be taught or suggested herein.

All of these implementations are intended to be within the scope of the devices, systems, and methods herein disclosed. These and other implementations will become readily apparent to those skilled in the art from the following detailed description of the implementations having reference to the attached figures, the devices, systems, and methods not being limited to any particular implementations disclosed.

In some implementations, an electronic device can include: a wearable interface including a first electrode and a second electrode spaced apart from the first electrode, the first and second electrodes electrically connected by conductive traces; and a first housing coupled to the wearable interface and couplable to an electronic component of the electronic device, the first housing including: a conductive bump configured to electrically contact the conductive traces; and a conductive post in electrical communication with the conductive bump, the conductive post providing electrical connection between a terminal of the electronic component and the first electrode and the second electrode.

In some implementations, the conductive post extends vertically relative to the first housing, the conductive post providing vertical electrical communication between the terminal and the second electrode. In some implementations, the wearable interface includes a patch configured to attach to a body of a user, the conductive traces disposed on or at least partially embedded in the patch. In some implementations, the first electrode is configured to transduce signals from the body of the user and transfer the signals to the electronic component via the conductive traces, the conductive bump in electrical contact with the conductive traces, and the conductive post in electrical contact with the conductive bump to the terminals of the electronic component.

In some implementations, the first housing includes a first housing body and a stiffener, wherein the conductive bump and the conductive post are disposed on the stiffener, and wherein the conductive post extends within a first hole in the wearable interface and a second hole in the first housing. In some implementations, the stiffener includes a polymer. In some implementations, the stiffener is formed using a two-shot injection molding process. In some implementations, the stiffener includes a base portion, the conductive post extending from the base portion. In some implementations, the stiffener includes a plurality of support posts extending from the base portion.

In some implementations, the conductive post and the plurality of support posts are formed of different materials. In some implementations, the conductive post includes a conductive polymer molded over the base portion. In some implementations, the conductive post electrically connects to a conductive inner boundary of the first hole in the wearable interface. In some implementations, the conductive post electrically connects to a horizontal segment of the wearable interface, the conductive post providing electrical communication vertically to an electrical terminal of the device. In some implementations, the electronic device includes an adhesive between the stiffener and the wearable interface. In some implementations, the electronic device includes a second housing connected to the first housing, the first housing or the second housing including the electronic component.

In some implementations, a method of forming an electronic device can include: providing a first housing including a base portion and a plurality of support posts extending from the base portion; and molding a conductive post on the base portion.

In some implementations, providing the first housing includes, before molding the conductive post, molding the base portion and the plurality of support posts. In some implementations, the method includes providing a wearable interface coupled with the first housing, the first housing couplable to an electronic component, wherein the wearable interface includes a first electrode and a second electrode spaced apart from the first electrode, the first electrode and the second electrode electrically connected via conductive traces.

In some implementations, the method includes extending the conductive post within a first hole in the wearable interface and a second hole in the first housing, the conductive post providing electrical connection between a terminal of the electronic component and the first electrode and the second electrode. In some implementations, molding the conductive post includes using a two-shot injection molding process, wherein a first shot forms the base portion and the plurality of support posts, and a second shot forms the conductive post from a conductive material. In some implementations, the method includes molding a conductive bump at a base of the conductive post.

### BRIEF DESCRIPTION OF THE DRAWINGS

These and other features, aspects, and advantages of the disclosure are described with reference to drawings of certain implementations, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 illustrates a schematic cross-section side view of an electronic device, according to various implementations of the present disclosure.

FIG. 2 illustrates a disassembled schematic cross-section side view of the electronic device of FIG. 1, according to various implementations of the present disclosure.

FIG. 3 illustrates an assembled schematic cross-section side view of the electronic device of FIG. 2, according to various implementations of the present disclosure.

FIG. 4 illustrates a schematic perspective view of a wearable interface coupled to the electronic device of FIGS. 1-3, according to various implementations of the present disclosure.

FIG. 5 illustrates a schematic perspective view of a stiffener coupled to the electronic device of FIGS. 1-3 and/or the wearable interface of FIG. 4, according to various implementations of the present disclosure.

FIG. 6 illustrates a schematic perspective view a first housing of the electronic device of FIGS. 1-3 coupled to the wearable interface of FIG. 4, according to various implementations of the present disclosure.

FIG. 7 illustrates a schematic cross-section side view of another electronic device, according to various implementations of the present disclosure.

FIG. 8 illustrates a schematic perspective view of a wearable interface of the electronic device of FIG. 7, according to various implementations of the present disclosure.

FIG. 9 illustrates a schematic perspective view of a first housing of the electronic device of FIG. 7, according to various implementations of the present disclosure.

### DETAILED DESCRIPTION

Although several implementations, examples, and illustrations are disclosed below, it will be understood by those of ordinary skill in the art that the devices, systems, and methods described herein extend beyond the specifically disclosed implementations, examples, and illustrations and includes other uses of the devices, systems, and methods and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of some specific implementations of the devices, systems, and methods. In addition, implementations can comprise several novel features. No single feature is solely responsible for its desirable attributes or is essential to practicing the devices, systems, and methods herein described.

The present disclosure may be understood by reference to the following detailed description. It is noted that, for purposes of illustrative clarity, certain elements in various drawings may not be drawn to scale, may be represented schematically or conceptually, or otherwise may not correspond exactly to certain physical configurations of implementations.

It can be beneficial to form wearable patch technologies using additive manufacturing methods, such as printed flexible substrate material (e.g., thermoplastic polyurethanes (TPUs), polyimide, acrylonitrile butadiene styrene (ABS), etc.) with printed biocompatible metals such as silver (Ag), silver chloride (AgCl) or other biocompatible conductors. In various implementations, low cost technologies can be used to form single metal layer with electrodes facing the human body (e.g., adhered over the chest). A disposable or reusable electric module can be positioned away from the body. The implementations disclosed herein can provide a low cost, robust vertical interconnect from the body side of the patch to the electronics side using a two-shot injection molded plastic or polymeric component with the second shot molded using a conductive polymer.

In addition, the two-shot molded part can comprise polypropylene and conductive thermoplastic polyurethane (TPU) to create compliant pads on the electronic side of the patch. This arrangement makes contact to a rigid metal target on the disposable or reusable electronic module (or nest). The compliance in the interconnect can preclude the need for pogo contacts or springs on the wearable device and can provide a fluid tight rating.

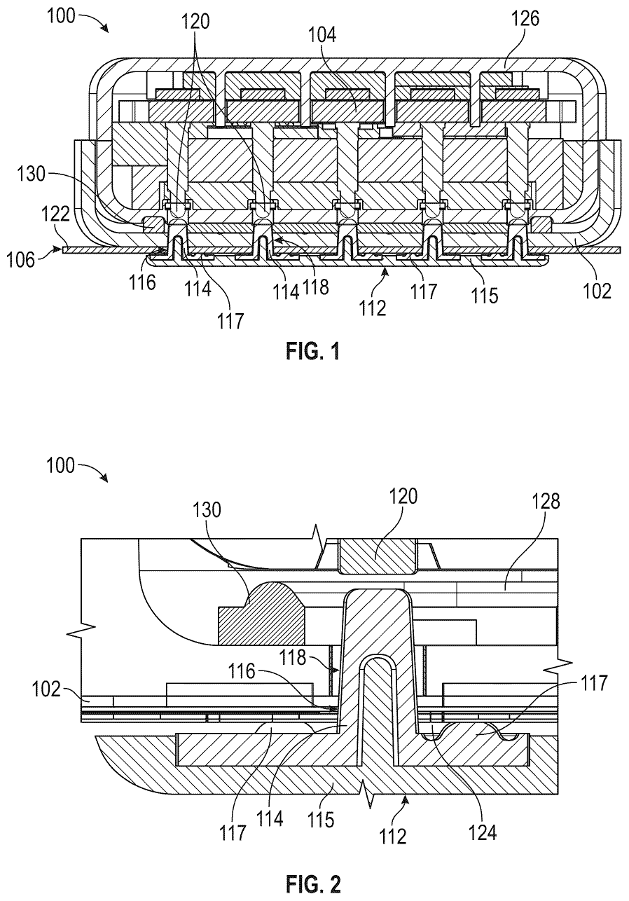

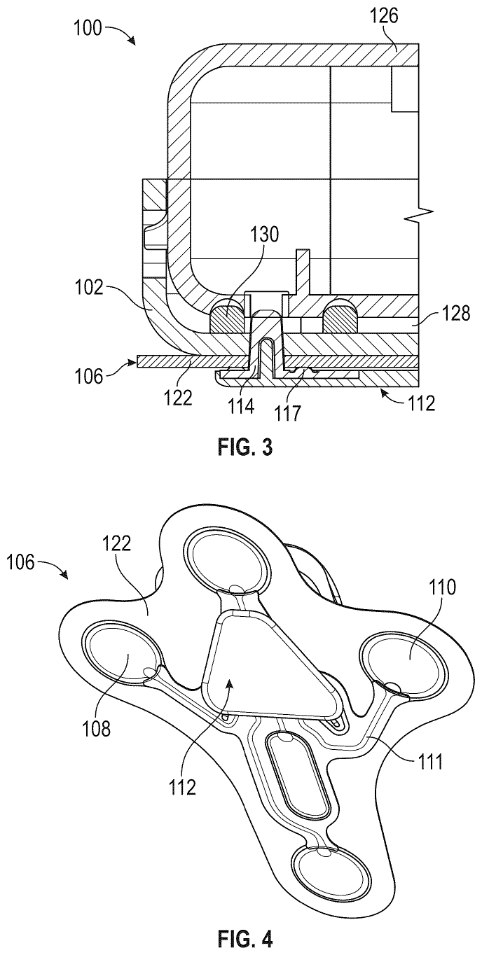

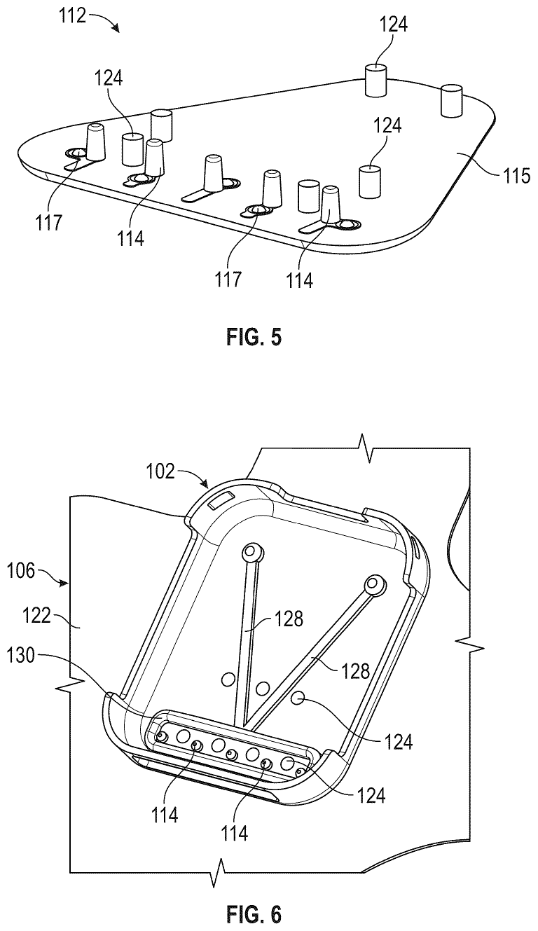

FIG. 1 illustrates a schematic cross-section side view of an example electronic device 100. FIG. 2 illustrates a disassembled schematic cross-section side view of the electronic device 100 of FIG. 1. FIG. 3 illustrates an assembled schematic cross-section side view of the electronic device 100 of FIG. 2. FIG. 4 illustrates a schematic perspective view of a wearable interface 106 of the electronic device 100 of FIGS. 1-3. FIG. 5 illustrates a schematic perspective view of a stiffener 112 coupled to the wearable interface 106 of the electronic device 100 of FIGS. 1-3 and/or the wearable interface of FIG. 4. FIG. 6 illustrates a schematic perspective view of the first housing 102 of the electronic device 100 of FIGS. 1-3 coupled to the wearable interface 106 of FIG. 4. In some implementations, the electronic device 100 can be a monitoring device (e.g., a sensor patch). The electronic device 100 can comprise a first housing 102 that can couple (e.g., mechanically fasten) to an electronic component 104 of the electronic device 100. The electronic component 104 can comprise any suitable type of electronic device, such as a sensor, an integrated device die, etc.

The electronic device 100 can further include a wearable interface 106 that can be coupled to the first housing 102. The wearable interface 106 can include a first electrode 108 and a second electrode 110 spaced apart from the first electrode 108. (see FIG. 4). The first electrode 108 and the second electrode 110 can be dry electrodes positioned on a skin surface of a user (e.g., a patient) to measure electrical potentials produced by the heart. The first electrode 108 and the second electrode 110 can be made from materials like silver/silver chloride, conductive polymers, carbon-based materials, metallic thin films, and/or conductive polymer composites that ensure conductivity and skin compatibility without gels. The first electrode 108 and the second electrode 110 can detect a physiological event (e.g., the electrical activity generated by cardiac muscle depolarization and repolarization). For example, the first electrode 108 and second electrode 110 can transduce the electrical potential differences on the skin caused by cardiac depolarization and repolarization, capturing signal voltages, frequency components, waveform morphology, amplitude variations, and timing intervals essential for cardiac monitoring and diagnosis. The signals from the electrodes 108, 110 can be converted from ionic currents from the heart into measurable electrical signals for further processing and analysis.

The first and second electrodes 108, 110 can be electrically connected by conductive traces 111. The conductive traces 111 can be comprised of any suitable conductive material, such copper, silver, gold, and/or conductive inks and pastes. In some implementations, the conductive traces 111 can be formed from silver material due to its high electrical conductivity and compatibility with flexible substrates used in wearable devices. The conductive traces 111 can extend from the electrodes 108, 110 to a structural stiffener 112, which provides mechanical support and stability to the flexible circuitry. As described below, the conductive traces 111 can interface with the stiffener 112 via conductive interfaces, such as thermoplastic elastomer (TPE) bumps or conductive posts, to facilitate reliable electrical connection.

The stiffener 112 can include one or more conductive bumps 117 that are positioned to make direct electrical contact with the conductive traces 111, which carry signals (e.g., bioelectrical signals) from the first and second electrodes 108, 110. The conductive bump 117 can serve as an electrical interface that facilitates a connection between the conductive traces 111 and the components of the first housing 102. The conductive bump 117 can effectively route the signals received from the electrodes 108, 110 through the conductive traces 111 to a conductive post 114 integrated with the stiffener 112. The conductive post 114 can extend vertically through both a first hole 116 formed in the wearable interface 106 and a second hole 118 formed in the first housing 102, thus electrically coupling the electrodes 108, 110 and the first housing 102. The conductive post 114 can serve as a rigid electrical conduit that carries the signals from the conductive bumps 117 upward to connect with other device elements. The positioning of the conductive post 114 within the holes 116, 118 can allow for mechanical stability and alignment within the first housing 102 while ensuring minimal signal loss or interference.

Together, the conductive post 114 and the conductive bump 117 can provide an electrical connection that couples a terminal 120 of the electronic component 104 and the first electrode 108 and/or the second electrode 110. This pathway can ensure that signals (e.g., low-amplitude cardiac bioelectrical signals) captured at the electrodes 108, 110 are transmitted with minimal resistance and noise to the electronic component 104. The combination of flexible conductive traces 111, the conductive bumps 117, and the conductive post 114 can enable electrical connectivity across the electronic device 100, including the wearable interface 106 (e.g., the flexible skin-contacting layers) and the first housing 102, thereby preserving signal integrity throughout the entire signal acquisition and transmission chain.

In some implementations, the wearable interface 106 comprises a patch 122 configured to attach to a body of the user (e.g., a patient), such as the chest or torso, for acquiring electrical signals. The conductive traces 111 can be disposed on or at least partially embedded in the patch 122 and serve as the signal routing elements. The patch 122 can be formed from a flexible, skin-safe substrate such as silicone, polyurethane, or a nonwoven fabric, allowing it to conform to the user's body while maintaining stable contact with the skin. The electrodes 108, 110, integrated into or affixed to the wearable interface 106, can be configured to transduce signals (e.g., bioelectrical signals) from the body of the user, such as signal associated with cardiac activity (e.g., a heart rate or any other suitable biological signal). The electrodes 108, 110 can convert the ionic signals present on the skin surface into electrical voltages that can be transmitted through the system for processing.

The conductive post 114 can electrically connect to different elements of the wearable interface 106. For example, the conductive post 114 can make electrical contact with a conductive inner boundary (e.g., inner radius) of the first hole 116 in the wearable interface 106, which can comprise a biocompatible conductor such as silver or silver chloride. The arrangement can allow for vertical signal transmission from the surface-level conductive traces 111, through the conductive bump 117 and into the conductive post 114, and toward the electronic component 104. It should be appreciated that the first and/or second holes 116, 118 can be any suitable shape, e.g., circular, elliptical, polygonal, etc. Further, the conductive post 114 can electrically connect to a horizontal segment of the wearable interface 106. As shown, a plurality of conductive posts 114 can extend from a base portion 115 of the stiffener 112 through the holes 116, 118.

In some implementations, as shown in FIG. 5, the stiffener 112 can formed from a polymer using a two-shot injection molding process. This two-step molding technique enables the creation of a multi-material or multi-component structure by sequentially injecting two distinct materials or layers into a mold. For example, in the first injection step, a base polymer is injected to form the foundational structure of the stiffener 112, including features such as the base portion 115 and the plurality of support posts 124. The base portion 115 and plurality of support posts 124 can provide structural reinforcement and positional stability. In the second injection step, a different material-such as a conductive polymer or a material with enhanced mechanical or electrical properties—is injected to form additional integrated conductive components, such as conductive posts 114 and/or conductive bumps 117, or to overmold previously formed features from the first shot. The resulting structure of the stiffener 112 following the two-shot process can include a visible or tactile material boundary at the interface between the first-shot material (e.g., ABS, PP, or PC of the base portion 115 and plurality of support posts 124) and the second-shot material (e.g., conductive TPE of the conductive post 114 and conductive bump 117), as well as alignment seams, interlocking geometries, or variations in surface texture or finish that may occur at or near the material interface.

As shown, the conductive post 114 extends vertically from the base portion 115 of the stiffener 112 and serves as an electrical interconnect between the lower layer of the wearable interface 106 (e.g., containing the electrodes 108, 110 and conductive traces 111) and the upper layer where the electronic component 104 are located. The conductive bump 117 can be formed (e.g., positioned) at the base of the conductive post 114 along the surface of the base portion 115. The conductive post 114 and conductive bump 117 can be formed as a single, integral structure during the second molding step, ensuring continuous electrical continuity and eliminating the need for separate assembly operations. Alternatively, in some implementations, the conductive post 114 and conductive bump 117 can be fabricated as separate components and assembled such that they maintain electrical contact, allowing greater material selection flexibility or modular construction.

In various implementations, the conductive post 114 and the plurality of support posts 124 are formed of different materials (e.g., different polymers) configured to perform separate electrical and mechanical functions. For example, the base portion 115 and/or the support posts 124 can be formed from a nonconductive polymer, such as acrylonitrile butadiene styrene (ABS), polypropylene (PP), polycarbonate (PC), high-density polyethylene (HDPE), or any other suitable material. The conductive post 114 and the conductive bump 117 can be formed from a conductive polymer material, such as a conductive thermoplastic elastomer (TPE) that includes conductive fillers, including but not limited to carbon black, silver particles, and/or graphite. Other suitable materials for the conductive elements include conductive silicone, conductive thermoplastic polyurethane (TPU), or thermoplastic vulcanizates (TPVs). This material differentiation enables the stiffener 112 to integrate mechanical support and electrical interconnect functionality, and can be implemented using multi-material molding processes, such as two-shot injection molding mentioned above.

This structural arrangement of the stiffener 112, conductive post 114, conductive bump 117, and/or plurality of support posts 124 can provide several advantages. First, by vertically integrating the conductive post 114 into the molded stiffener 112, electrical signals from the electrodes 108, 110 can be transmitted along the conductive traces 111 to the conductive post 114 with minimal signal loss and without requiring external wires, soldering, or manual assembly, thereby improving reliability and reducing manufacturing complexity. Second, the alignment provided by the conductive post 114, support posts 124, and base portion 115 can ensure the positioning of the conductive elements relative to the surrounding housing and wearable interface is correct, supporting mechanical integrity during repeated use or flexing. Third, forming the conductive bump 117 as part of or in conjunction with the conductive post 114 simplifies the electrical connectivity layout and ensures consistent electrical contact with the conductive traces 111. Additionally, the two-shot molding process allows for the use of optimized materials in each region, such as using a mechanically strong but non-conductive polymer for the stiffener 112 and base portion 115 and a conductive, biocompatible material for the conductive post 114 and conductive bump 117, resulting in an integrated, durable, and electrically functional interconnect solution within a compact wearable form factor.

As mentioned above, the stiffener 112 can comprise a plurality of support posts 124 extending from the base portion 115. The support posts 124 can provide structural support to the electronic device 100 during use by maintaining the relative positioning of the stiffener 112 in relation to the wearable interface 106. In some implementation, the plurality of support posts 124 are heat stake pillars, which are small plastic post molded into the base portion 115, and designed to be deformed using heat to mechanically couple the stiffener 112 to the patch 122. Each heat stake pillar can be formed as an integral extension of the base portion 115 during the molding process. The heat stake pillars are configured to extend through corresponding openings in the patch 122. During assembly, the exposed ends of the heat stake pillars can be locally heated and deformed to form a mechanical head or cap that secures the stiffener 112 to the patch 122. This heat staking process creates a mechanical bond without the use of adhesives, fasteners, or inserts, and can provide a permanent or semi-permanent attachment depending on the material properties and deformation parameters. The use of heat stake pillars allows for mechanical coupling between the stiffener 112 and surrounding layers, reducing assembly complexity and maintaining alignment under mechanical stress, movement, or flexing during operation of the electronic device 100. The heat stake pillars can be formed from the same nonconductive polymer as the base portion 115, such as ABS, PP, or PC, or may be composed of a different thermoplastic material compatible with thermal deformation and the intended mechanical function.

An adhesive can be provided between the stiffener 112 and the wearable interface 106 to secure the wearable interface 106 and stiffener together 112, and to provide a watertight seal over the electrodes 108, 110. The adhesive can be applied in one or more regions of contact between the stiffener 112 and the patch 122 of the wearable interface 106. The adhesive can contribute to the retention and alignment of the stiffener 112 relative to the wearable interface 106. The adhesive can also form a continuous barrier to protect features of the electronic device 100, such as the conductive post 114 and/or conductive bump 117, from moisture, sweat, debris, or other contaminants that may interfere with signal acquisition or degrade material performance over time. The adhesive can be a pressure-sensitive adhesive, a heat-activated adhesive, a UV-curable adhesive, a silicone-based adhesive, or any other biocompatible adhesive formulation suitable for use in skin-contacting medical or wearable devices.

As shown in FIGS. 1 and 3, a second housing 126 can be connected to the first housing 102. The second housing 126 can be mechanically coupled to the first housing 102 through one or more fastening features, such as snap fits, ultrasonic welding, mechanical clips, or adhesive bonding. The first or second housings 102, 126 can include the electronic component 104, such as sensor electronics, a processor, a battery, etc. In the illustrated implementations, the upper second housing 126 can comprise the electronic component(s) 104 which can electrically connect to the conductive post(s) 114 by way of terminals 120 connected to the electronic component(s) 104.

In some implementations, as shown in FIG. 6, the first housing 102 can include a vibration dampener 128 extending along a length of the first housing 102. The vibration dampener 128 can be positioned between the first housing 102 and second housing 126 to reduce transmission of mechanical vibrations between the housings 102, 126. The vibration dampener 128 can absorb or dissipate vibrational energy generated during operation of the electronic device 100 or external movement, thereby protecting sensitive internal components and improving signal stability. At a first end, the vibration dampener 128 can terminal at a seal 130 that encircles the conductive post 114. The seal 130 can provide environmental protection by preventing ingress of moisture, dust, or other contaminants, and can provide electrical isolation around the conductive post 114 to reduce risk of short circuits or interference. In some implementations, the vibration dampener 128 and scal 130 can comprise the same material. For example, the vibration dampener 128 and/or seal 130 can be formed from elastomeric or viscoelastic materials, such as silicone rubber, thermoplastic elastomers (TPE), or other suitable polymers with vibration damping and sealing properties.

The electronic component 104 housed within the second housing 126 can include one or more integrated circuits configured to receive the electrical signals routed from the electrodes 108, 110 via the conductive post 114 and conductive bumps 117. The component 104 can perform functions including signal amplification, filtering, analog-to-digital conversion, and data processing. Signal amplification increases the amplitude of the low-level bioelectrical signals to levels suitable for further processing and analysis. Filtering removes noise and artifacts outside the target frequency range of the cardiac signals. The analog-to-digital converter (ADC) converts the conditioned analog signals into digital data for processing by a microcontroller or digital signal processor within the component 104. The component 104 may further include memory for data storage and wireless communication modules for transmitting processed data to external devices such as smartphones or medical monitoring systems. The component 104 can be electrically connected to the conductive post 114 via terminals 120, enabling the signal transfer while maintaining electrical isolation and environmental protection provided by the surrounding housing and seal structures.

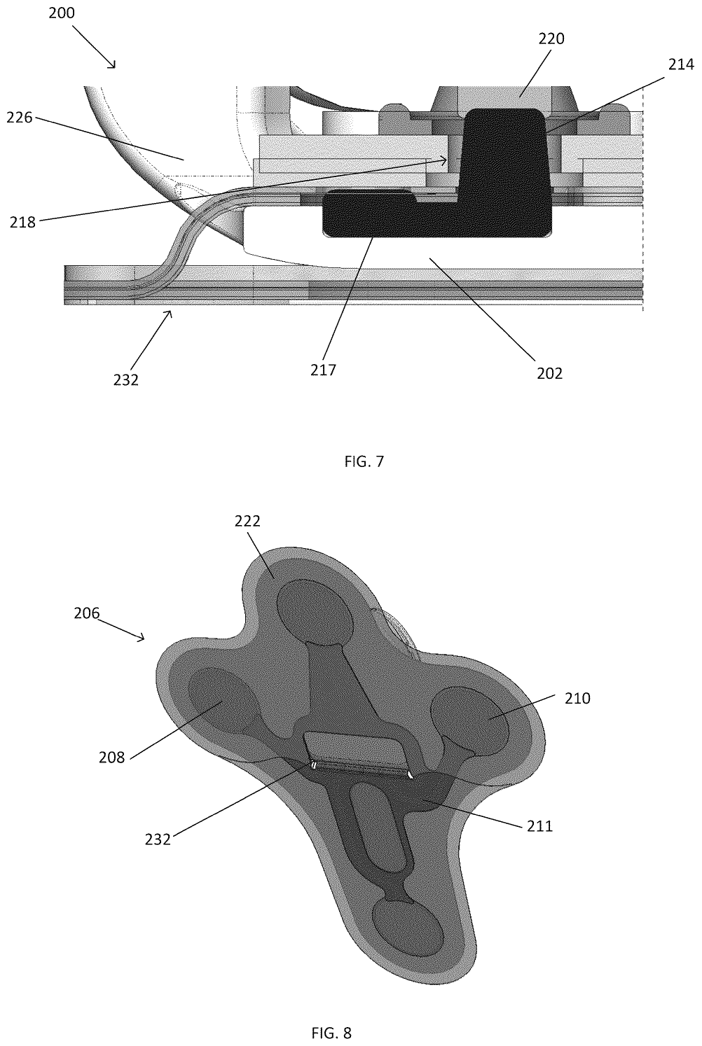

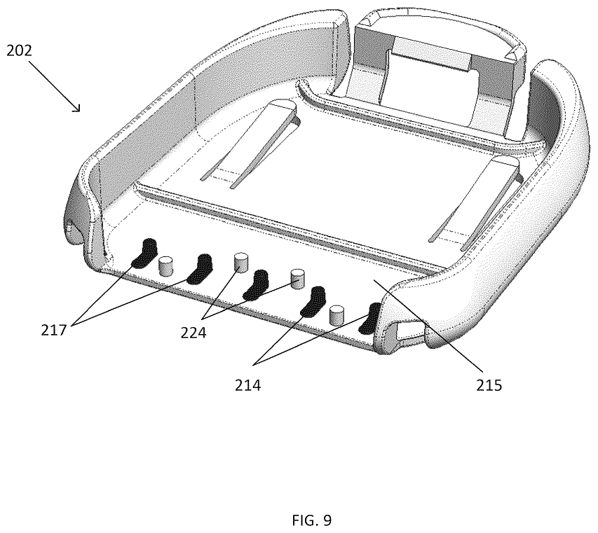

FIG. 7 illustrates a schematic cross-section side view of an example electronic device 200. FIG. 8 illustrates a schematic perspective view of a wearable interface 206 of the electronic device 200 of FIG. 7. FIG. 9 illustrates a schematic perspective view of the first housing 202 of the electronic device 200 of FIG. 7 couplable to the wearable interface 206 of FIG. 8. Unless otherwise noted, the components of FIGS. 7-9 can be the same as or generally similar to like-numbered components of FIGS. 1-6, where reference numbers for corresponding components are incremented by multiples of 100. As illustrated in FIGS. 7-9, the electronic device 200 can include a first housing 202, a wearable interface 206, a first electrode 208, a second electrode 210, conductive traces 211, a conductive post 214, a base 215, a conductive bump 217, a hole 218, terminals 220, a patch 122, a plurality of support posts 224, and a second housing 226.

The electronic device 200 can include a wearable interface 206 that can be coupled to the first housing 202. The wearable interface 206 can include a first electrode 208 and a second electrode 210 spaced apart from the first electrode 208. (see FIG. 8). A second housing 226 can be coupled to the first housing 202. The second housing 226 can be mechanically coupled to the first housing 102 through one or more fastening features, such as snap fits, ultrasonic welding, mechanical clips, or adhesive bonding. The first housings 202 or second housing 226 can include an electronic component, such as sensor electronics, a processor, a battery, etc. For example, the upper second housing 226 can comprise the electronic component which can electrically connect to the conductive post 214 by way of the terminals 220 connected to the electronic component.

The first housing 202 can include the conductive post 214 and the conductive bumps 217. The conductive traces 211, which carry signals (e.g., bioelectrical signals) from the first and second electrodes 208, 210, of the wearable interface 206 can be in electrical communication with the conductive bump 217. The conductive bump 217 can serve as an electrical interface to prove a connection between the conductive traces 211 and the components of the first housing 202. The conductive bump 217 can route the signals to the conductive post 214 integrated with the first housing 202. The conductive post 214 can extend vertically through a hole 218 formed in the second housing 226, thus electrically coupling the electrodes 208, 210 and the second housing 226. The conductive post 214 can serve as a rigid electrical conduit that carries the signals from the conductive bumps 217 upward to connect with other device elements.

As shown in FIGS. 7 and 8, the electronic device 200 can further include an aperture 232 (e.g., a flap) through the wearable interface 206 (e.g., from the body side of the patch 222 to the electronics side), through which the conductive traces 211 can be routed to electrically couple the electrodes 208, 210 to the conductive post 214 and the conductive bump 217. The conductive traces 211 can be disposed on or embedded within the patch 222 of the wearable interface 206, and can be formed from a conductive material such as silver, copper, or gold. The conductive traces 211 can provide low-resistance pathways for transmitting bioelectrical signals from the electrodes 208, 210 to the conductive bump 217, which serves as an electrical interface between the flexible wearable interface 206 and the components of the device 200 coupled to the first housing 202. The aperture 232 can be shaped to accommodate trace routing, alignment features, and/or manufacturing constraints, and be circular, elliptical, polygonal, or any other geometry suitable for the configuration of the device and the materials used. The aperture 232 can ensure that the skin of the user does not contact rigid materials, such as plastics.

In some implementations, as shown in FIG. 8, the wearable interface 206 comprises a patch 222 configured to attach to a body of the user, such as the chest or torso, for the purpose of acquiring bioelectrical signals. The patch 222 can be secured to the skin using a skin-safe adhesive, and can maintain contact between the electrodes 208, 210 and the user's skin during motion or extended wear. The entirety or a substantial portion of the patch 222 can be in contact with the user's body, providing a broad area for mechanical stability and consistent signal acquisition. The electrodes 208, 210 can be positioned on the patch in a spaced arrangement to detect electrical potentials associated with cardiac activity, which are then routed via the conductive traces 211 to the conductive bump 217 and conductive post 214 to the electrical component.

As shown in FIG. 9, the conductive post 214 and the conductive bump 217 can be integrated into or onto the first housing 202 at a base 215, along with a plurality of support posts 224. The conductive post 214 and conductive bump 217 can be formed from a conductive polymer, such as a conductive thermoplastic elastomer (TPE), containing conductive fillers such as carbon black, silver particles, or graphite. The first housing 202 and support posts 224 can be formed from a nonconductive polymer such as acrylonitrile butadiene styrene (ABS), polypropylene (PP), or polycarbonate (PC), and serve to mechanically support the housing structure and maintain alignment between the first housing 202 and the second housing. In some implementations, the conductive post 214 and support posts 224 can be manufactured using a two-shot injection molding process, where the first shot forms the base structure (e.g., the first housing 202) including the support posts 224 and the second shot overmolds the conductive material to form the conductive post 214 and the conductive bump 217. This process allows for secure integration of mechanical and electrical components, with material boundaries or surface transitions at the interface serving as indicators of the two-shot molding process. The support posts 224 can further function as heat stake pillars, which can be thermally deformed to secure the first housing 202 to the second housing. The conductive post 214 can interface with the terminal 220 of the electronic component housed in the second housing and configured to amplify, filter, digitize, and process the acquired signals for transmission or storage.

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain implementations and examples, it will be understood by those skilled in the art that the various implementations of the systems and processes extend beyond the specifically disclosed implementations to other alternative implementations and/or uses of the systems and processes and obvious modifications and equivalents thereof. In addition, while several variations of the implementations of the systems and processes have been shown and described in detail, other modifications, which are within the scope of this disclosure, will be readily apparent to those of skill in the art based upon this disclosure. It is also contemplated that various combinations or sub-combinations of the specific features and implementations of the implementations may be made and still fall within the scope of the disclosure. It should be understood that various features and implementations of the disclosed implementations can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative implementations, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate implementations also may be implemented in combination in a single implementations. Conversely, various features that are described in the context of a single embodiment also may be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Moreover, as used herein, when a first element is described as being “on” or “over” a second element, the first element may be directly on or over the second element, such that the first and second elements directly contact, or the first element may be indirectly on or over the second element such that one or more elements intervene between the first and second elements. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain implementations include, while other implementations do not include, certain features, elements and/or states. Thus, such conditional language is not generally intended to imply that features, elements and/or states are in any way required for one or more embodiments.

While certain implementations have been described, these implementations have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative implementations may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further implementations. The accompanying claims and their equivalents are intended to cover such forms or modifications as would fall within the scope and spirit of the disclosure.

Several illustrative examples of injection molded interconnects and related systems and methods have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps may be arranged or performed differently than described and components, elements, features, acts, or steps may be combined, merged, added, or left out in various examples. All possible combinations and subcombinations of elements and components described herein are intended to be included in this disclosure. No single feature or group of features is necessary or indispensable.

Certain features that are described in this disclosure in the context of separate implementations may also be implemented in combination in a single implementation. Conversely, various features that are described in the context of a single implementation also may be implemented in multiple implementations separately or in any suitable subcombination. Moreover, although features may be described above as acting in certain combinations, one or more features from a claimed combination may in some cases be excised from the combination, and the combination may be claimed as a subcombination or variation of a subcombination.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures may or may not be drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components may be added, removed, and/or rearranged. Further, the disclosure herein of any particular feature, aspect, method, property, characteristic, quality, attribute, element, or the like in connection with various examples may be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures, or description herein. For example, various functionalities provided by the illustrated modules may be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification may be included in any example.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the implementations shown herein but are to be accorded a fair interpretation consistent with this disclosure, the principles and the novel features disclosed herein.

What is claimed is:

1. An electronic device comprising:
a wearable interface comprising a first electrode and a second electrode spaced apart from the first electrode, the first electrode and the second electrode electrically connected by conductive traces; and
a first housing coupled to the wearable interface and couplable to an electronic component of the electronic device, the first housing comprising:
a conductive bump configured to electrically contact the conductive traces; and
a conductive post in electrical communication with the conductive bump, the conductive post providing electrical connection between a terminal of the electronic component and the first electrode and the second electrode.

2. The electronic device of claim 1, wherein the conductive post extends vertically relative to the first housing, the conductive post providing vertical electrical communication between the terminal and the second electrode.

3. The electronic device of claim 1, wherein the wearable interface comprises a patch configured to attach to a body of a user, the conductive traces disposed on or at least partially embedded in the patch.

4. The electronic device of claim 3, wherein the first electrode is configured to transduce signals from the body of the user and transfer the signals to the electronic component via the conductive traces, the conductive bump in electrical contact with the conductive traces, and the conductive post in electrical contact with the conductive bump to the terminal of the electronic component.

5. The electronic device of claim 1, wherein the first housing comprises a first housing body and a stiffener, wherein the conductive bump and the conductive post are disposed on the stiffener, and wherein the conductive post extends within a first hole in the wearable interface and a second hole in the first housing.

6. The electronic device of claim 5, wherein the stiffener comprises a polymer.

7. The electronic device of claim 6, wherein the stiffener is formed using a two-shot injection molding process.

8. The electronic device of claim 6, wherein the stiffener comprises a base portion, the conductive post extending from the base portion.

9. The electronic device of claim 8, wherein the stiffener comprises a plurality of support posts extending from the base portion.

10. The electronic device of claim 9, wherein the conductive post and the plurality of support posts are formed of different materials.

11. The electronic device of claim 10, wherein the conductive post comprises a conductive polymer molded over the base portion.

12. The electronic device of claim 5, wherein the conductive post electrically connects to a conductive inner boundary of the first hole in the wearable interface.

13. The electronic device of claim 12, wherein the conductive post electrically connects to a horizontal segment of the wearable interface, the conductive post providing electrical communication vertically to an electrical terminal of the electronic component.

14. The electronic device of claim 5, further comprising an adhesive between the stiffener and the wearable interface.

15. The electronic device of claim 1, further comprising a second housing connected to the first housing, the first housing or the second housing comprising the electronic component.

16. A method of forming an electronic device, the method comprising:
providing a first housing comprising a base portion and a plurality of support posts extending from the base portion; and
molding a conductive post on the base portion.

17. The method of claim 16, wherein providing the first housing comprises, before molding the conductive post, molding the base portion and the plurality of support posts.

18. The method of claim 16, further comprising providing a wearable interface coupled with the first housing, the first housing couplable to an electronic component, wherein the wearable interface comprises a first electrode and a second electrode spaced apart from the first electrode, the first electrode and the second electrode electrically connected via conductive traces.

19. The method of claim 18, further comprising extending the conductive post within a first hole in the wearable interface and a second hole in the first housing, the conductive post providing electrical connection between a terminal of the electronic component and the first electrode and the second electrode.

20. The method of claim 16, wherein molding the conductive post comprises using a two-shot injection molding process, wherein a first shot forms the base portion and the plurality of support posts, and a second shot forms the conductive post from a conductive material.

21. The method of claim 16, further comprising molding a conductive bump at a base of the conductive post.
